(19) United States
(12) Patent Application Publication
(10) Pub. No.: US 2019/0019033 A1
(43) Pub. Date: Jan. 17, 2019

(54) APPARATUS AND METHOD FOR GENERATING OLFACTORY INFORMATION RELATED TO MULTIMEDIA CONTENT

(71) Applicant: Electronics and Telecommunications Research Institute, Daejeon (KR)

(72) Inventors: Sung June CHANG, Daejeon (KR); Hae Ryong LEE, Daejeon (KR); Jun Seok PARK, Daejeon (KR); Joon Hak BANG, Sejong-si (KR); Jong Woo CHOI, Daejeon (KR); Sang Yun KIM, Daejeon (KR); Hyung Gi BYUN, Seoul (KR); Jang Sik CHOI, Donghae-si (KR)

(73) Assignee: Electronics and Telecommunications Research Institute, Daejeon (KR)

(21) Appl. No.: 15/822,376

(22) Filed: Nov. 27, 2017

(30) Foreign Application Priority Data
Jul. 13, 2017 (KR) 10-2017-0089197

(51) Int. Cl. G06K 9/00; G06K 9/72; G06K 9/62; G10L 25/03; G10L 25/27; G10L 25/72; G01N 33/00

(52) U.S. Cl. CPC G06K 9/00671; G06K 9/00744; G06K 9/00718; G06K 9/726; G06K 9/6289; G06F 17/2785; G10L 25/27; G10L 25/72; G01N 33/0004; G01N 33/0062; G10L 25/03

(57) ABSTRACT

An apparatus for generating olfactory information related to multimedia content may comprise a processor. The processor may receive multimedia content, extract an odor image or an odor sound included in the multimedia content, and generate representative data related to the odor image or the odor sound by describing information on the extracted odor image or odor sound in a data format sharable by a media thing.

### CLAIM FOR PRIORITY

This application claims priority to Korean Patent Application No. 2017-0089197 filed on Jul. 13, 2017 in the Korean Intellectual Property Office (KIPO), the entire contents of which are hereby incorporated by reference.

### BACKGROUND

### 1. Technical Field

The present invention relates to a method of representing an ability of an electronic nose apparatus and transmission of a recognized scent in a virtual reality system based on Internet of Media Things and Wearables (IoMT), and more particularly, to IoMT technology for providing interoperability between a virtual world and the real world in a virtual reality system.

### 2. Related Art

The present invention relates to a method of representing an ability of an electronic nose apparatus and transfer of a recognized scent in a virtual reality system based on Internet of Media Things and Wearables (IoMT), and more particularly, to an IoMT technology for providing interoperability between a virtual world and the real world in a virtual reality system.

The concept of an electronic nose (E-nose) is used for a sensor which senses particles or gases which cause a scent in the real world. In the real world, a scent is sensed using a physical, chemical, or biological method and on the basis of a concentration of a gas or a concentration of particles which cause the scent.

A method of representing olfactory information sensed by an E-nose sensor to reproduce it in a virtual world or the real world has been attempted through the Conference on the Standardization of IoMT.

This is the time at which it is necessary to develop a data type for sharing olfactory information between a virtual world and real world, which is advanced and standardized through Conference on the Standardization of IoMT as described above.

### SUMMARY

Accordingly, example embodiments of the present invention are provided to substantially obviate one or more problems due to limitations and disadvantages of the related art.

Example embodiments of the present invention provide an apparatus for generating olfactory information related to multimedia content.

Example embodiments of the present invention also provide a method for generating olfactory information related to multimedia content.

In order to achieve the objective of the present disclosure, an olfactory information generator, which generates olfactory information sharable between the real world and at least one virtual world, may comprise a processor and the processor may receive multimedia content, extract an odor image or an odor sound included in the multimedia content, and generate representative data related to the odor image or the odor sound by describing information on the extracted odor image or odor sound in a data format sharable by a media thing.

The processor may analyze the extracted odor image or odor sound and generate text-cased label information capable of describing an odor of the odor image or the odor sound through a semantic evaluation or an abstract process related to the analyzed odor image or odor sound.

The processor may update the label information of the extracted odor image or odor sound by applying a pattern recognition technique to odor image or odor sound data included in a database related to the extracted odor image or odor sound.

The processor may extract each of a plurality of odor images or odor sounds included in the multimedia content and generate the representative data by using information on each of the plurality of extracted odor images or odor sounds, with a weight.

The processor may generate the representative data by using synchronization information between the extracted odor image or odor sound and the multimedia content to form a scent emitting sequence corresponding to the odor image or the odor sound to be synchronized with execution of the multimedia content.

The processor may receive sensory information related to a scent in the real world, which is generated by a gas sensor, extract odor image or odor sound information related to content of the multimedia content, which is time-synchronized with the sense information, and generate the representative data by adding the sensory information to the extracted odor image or odor sound information extracted related to the content time-synchronized with the sensory information.

In order to achieve the objective of the present disclosure, an olfactory information generator, which generates olfactory information sharable between a real world and at least one virtual world, may comprise a processor and the processor may obtain text-based label information related to a scent component included in the scent cartridge and generate representative data related to the label information by describing information on the label information related to the scent component in a data format sharable by a media thing.

The processor may search an odor information-odor image association database, an odor information-odor sound association database, or an odor information-label information association database for the scent component and extract the label information corresponding to the scent component.

The processor may obtain the label information by a user input, extract modified label information corresponding to the label information by searching an odor information-odor image association database, an odor information-odor sound association database, or an odor information-label information association database for the label information, and generate the representative data in connection with the label information and the modified label information.

The processor, periodically or when a particular event occurs, may execute searching of an odor information-odor image association database, an odor information-odor sound association database, or an odor information-label information association database, execute pattern recognition or text syntax analysis, and updates the label information.

In order to achieve the objective of the present disclosure, a method, which is generating olfactory information in which olfactory information sharable between a real world and at least one virtual world is generated, may comprise receiving multimedia content, extracting an odor image or an odor sound included in the multimedia content, and describing information on the extracted odor image or odor sound in a data format sharable by a media thing.

In order to achieve the objective of the present disclosure, a method, which is generating olfactory information in which olfactory information sharable between a real world and at least one virtual world is generated, may comprise identifying whether a scent cartridge comprising a scent component is equipped, obtaining text-based label information related to the scent component, and generating representative data related to the label information by describing the label information related to the scent component in a data format sharable by a media thing.

In order to achieve the objective of the present disclosure, a computer-readable recording medium in which a program for executing the method according to any one of above methods is recorded.

According to the embodiments of the present invention, interoperability between a virtual world and the real world may be provided by recognizing an odor which exists in the real world within a range of IoMT and transmitting the odor of the real world to the virtual world.

The present invention is a configuration which digitalizes and represents types of odors sensed by an actual olfactory sense, a time necessary for sensing, fatigability of an olfactory organ of a human body, and the like to correspond to an action of a real human body's olfactory organ. Through this, it is possible to accelerate commercialization of research on digitalizing the five senses of a human for things such as virtual reality, olfactory displays, scent displays, or the like.

According to the embodiments of the present invention, detailed information may be generated and transmitted during a process of transmitting an odor in the real world to a virtual world. According to the embodiments of the present invention, information related to an olfactory sense may be extracted from multimedia content and may be provided in a format interoperable between a virtual world and the real world or between virtual worlds.

According to the embodiments of the present invention, multimedia content and a data format capable of sharing olfactory information related to the multimedia content may be provided by using a connection between a media thing having a media function and a server or between media things.

According to the embodiments of the present invention, olfactory information related to multimedia content may be reproduced to be more similar to reality by analyzing a scent component included in a scent cartridge meant to reproduce shared olfactory information.

### BRIEF DESCRIPTION OF DRAWINGS

Example embodiments of the present invention will become more apparent by describing in detail example embodiments of the present invention with reference to the accompanying drawings, in which:

FIG. 1 is a view illustrating an overall execution environment including an olfactory information generator according to one embodiment of the present invention;

FIG. 2 is a view illustrating a process of analyzing or recognizing an odor image matched with a scent component equipped in a scent cartridge and generating and updating label information which represents the scent component;

FIG. 3 is a view illustrating, as an example of a process of embodying the scent display or the olfactory display, a process of extracting an odor image which associates a scent with image content, representing the odor image as label information, and sharing the label information with a scent display or an olfactory display and emitting a scent;

FIG. 4 is a view illustrating an example of a data format which represents label information of an odor image;

FIG. 5 is a view illustrating an example of a binary representation of the label information of FIG. 4;

FIG. 6 is a view illustrating an example of representative data which represents whether the olfactory information generator or a media thing (Mthing) has a function of recognizing label information of an odor image from the odor image;

FIG. 7 is a view illustrating an example of a binary representation of the representative data of FIG. 6;

FIG. 8 is a view illustrating an example of representative data which represents a command for controlling (an)other media thing(s) so that the other media thing(s) allow(s) the olfactory information generator or the media thing(s) to recognize label information of an odor image;

FIG. 9 is a view illustrating an example of a binary representation of the representative data of FIG. 8;

FIG. 10 is a view illustrating an example of a schema diagram of representative data which represents a recognition function of a processor, the olfactory information generator, or a media thing which recognizes an odor image or label information of the odor image;

FIG. 11 is a view illustrating an example of a syntax structure of representative data which represents a recognition function of a processor, the olfactory information generator, or a media thing which recognizes an odor image or label information of the odor image;

FIGS. 12 and 13 are views illustrating semantics of representative data which represents a recognition function of recognizing an odor image or label information of the odor image;

FIG. 14 is a view illustrating an example of representative data to which the syntax structure of FIG. 11 and the semantics of FIGS. 12 and 13 are applied;

FIG. 15 is a view illustrating an example of a schema diagram of representative data which represents a recognition command for a processor, the olfactory information generator, or a media thing which recognizes an odor image or label information of the odor image;

FIG. 16 is a view illustrating an example of a syntax structure of representative data which represents a recognition command for a processor, the olfactory information generator, or a media thing which recognizes an odor image or label information of the odor image;

FIG. 17 is a view illustrating example semantics of representative data which represents a recognition command for a processor, the olfactory information generator, or a media thing which recognizes an odor image or label information of the odor image;

FIG. 18 is a view illustrating an example of representative data to which the syntax structure of FIG. 16 and the semantics of FIG. 17 are applied;

FIG. 19 is a view illustrating an example of a schema diagram of representative data which represents a result of recognizing an odor image or label information of the odor image using the olfactory information generator or a media thing;

FIG. 20 is a view illustrating an example of a syntax structure of representative data which represents a result of recognizing an odor image or label information of the odor image using the olfactory information generator or a media thing;

FIG. 21 is a view illustrating an example of semantics of representative data which represents a result of recognizing an odor image or label information of the odor image by the olfactory information generator or a media thing;

FIG. 22 is a view illustrating an example of representative data to which the syntax structure of FIG. 20 and the semantics of FIG. 21 are applied;

FIG. 23 is a view illustrating an example of a schema diagram of representative data handled in a system environment including an olfactory display or a scent display;

FIG. 24 is a view illustrating an example of a syntax structure of representative data handled in a system environment including the olfactory display or the scent display;

FIG. 25 is a view illustrating an example of semantics of representative data handled in a system environment including the olfactory display or the scent display; and

FIG. 26 is a view illustrating an example of representative data to which the syntax structure of FIG. 24 and the semantics of FIG. 25 are applied.

### DESCRIPTION OF EXAMPLE EMBODIMENTS

Embodiments of the present disclosure are disclosed herein. However, specific structural and functional details disclosed herein are merely representative for purposes of describing embodiments of the present disclosure, however, embodiments of the present disclosure may be embodied in many alternate forms and should not be construed as limited to embodiments of the present disclosure set forth herein.

Accordingly, while the present disclosure is susceptible to various modifications and alternative forms, specific embodiments thereof are shown by way of example in the drawings and will herein be described in detail. It should be understood, however, that there is no intent to limit the present disclosure to the particular forms disclosed, but on the contrary, the present disclosure is to cover all modifications, equivalents, and alternatives falling within the spirit and scope of the present disclosure. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular embodiments only and is not intended to be limiting of the present disclosure. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the present disclosure will be described in greater detail with reference to the accompanying drawings.

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein.

A general virtual world processing system included as a part of a configuration of the present invention may correspond to an engine, a virtual world, and the real world. In the real world, an electronic nose (E-nose) apparatus senses information related to the real world or a scent emitting device embodies information related to a virtual world in the real world. Also, the virtual world may include a virtual world itself embodied by a program or a scent media reproducer which reproduces content including scent-emitting information capable of being embodied in the real world.

For example, a scent in the real world, information on abilities and data of the E-nose apparatus, and the like may be sensed and transmitted to an engine by the E-nose apparatus. Also, the E-nose apparatus may include an E-nose Capability Type which transfers the abilities and data of the E-nose apparatus to the engine, an Odor Sensor Technology Classification Scheme which describes a type of sensor necessary for definition of the E-nose Capability Type, and an Enose Sensed Info Type which transfers information recognized by the E-nose apparatus to the engine.

The engine may transmit sensed information to a virtual world. Here, the sensed information is applied to the virtual world such that an effect corresponding to the Enose sensed info type corresponding to a scent of the real world may be embodied in the virtual world.

An effect event which occurs in the virtual world may be driven by the scent emitting device of the real world. Virtual information (sensory effects) related to the effect event which occurs in the virtual world may be transmitted to the engine. Also, virtual world object characteristics may be mutually transmitted between the virtual world and the engine.

The scent emitting device which exists in the real world and accommodates user preference will be described in the realm of Internet of Media Things and Wearables (IoMT). The scent emitting device exists in the real world and emits a scent to a user to allow the user to be synchronized with content of the virtual world and to have a realistic experience. For this, that which transfers the abilities and data of the scent emitting device to the engine is referred to as a Scent Capability Type. Also, that which accommodates a preference of the user to compensate for a difference in characteristics of a scent provided by the scent emitting device and a scent sensed by the user is referred to as a Scent Preference Type. Also, that which commands in order to allow the scent emitting device to emit a scent is referred to as a scent effect.

A generalized virtual world processing method included as a part of a configuration of the present invention may be performed by mutually transmitting olfactory information between a virtual world, the real world, and another virtual world to represent the olfactory information through the scent emitting device. The generalized virtual world processing method may obtain virtual information which is olfactory information of the virtual world, obtain real information that is olfactory information of the real world through a reality recognizer which is an apparatus which recognizes a scent, provide the virtual information to the real world or the other virtual world, provide the real information to the virtual world or the other virtual world, and emit a scent to a user through a scent emitting device on the basis of the virtual information and the real information.

The real information includes a type of sensor necessary for defining the E-nose Capability Type which transfers the abilities and data of the E-nose apparatus which is the reality recognizer, the Scent Sensor Technology CS, information recognized by the E-nose, and the Enose Sensed Info Type which is a part which transfers the information recognized by the Enose.

Also, an operation of defining the Scent Capability Type, which transfers the ability and data of the scent emitting device which emits a scent to the engine, an operation of defining a Scent Preference Type which transfers a user preference to compensate for a difference in characteristics of a scent provided by the scent emitting device and a scent sensed by the user, and an operation of defining a Scent Effect which commands in order to allow the scent emitting device to emit a scent are included.

The terms “a scent display” or “an olfactory display” stated herein adds a scent to content and provides the user with the scent-added content while interworking with, for example, a personal computer, a laptop computer, a mobile terminal, a television, or an audiovisual display such as a head mounted display (HMD) and the like. The scent display or the olfactory display may include a scent cartridge which includes a scent component and may further include a controller or a processor which controls the scent cartridge to embody a scent atmosphere by discharging the scent component or a combination of scent components.

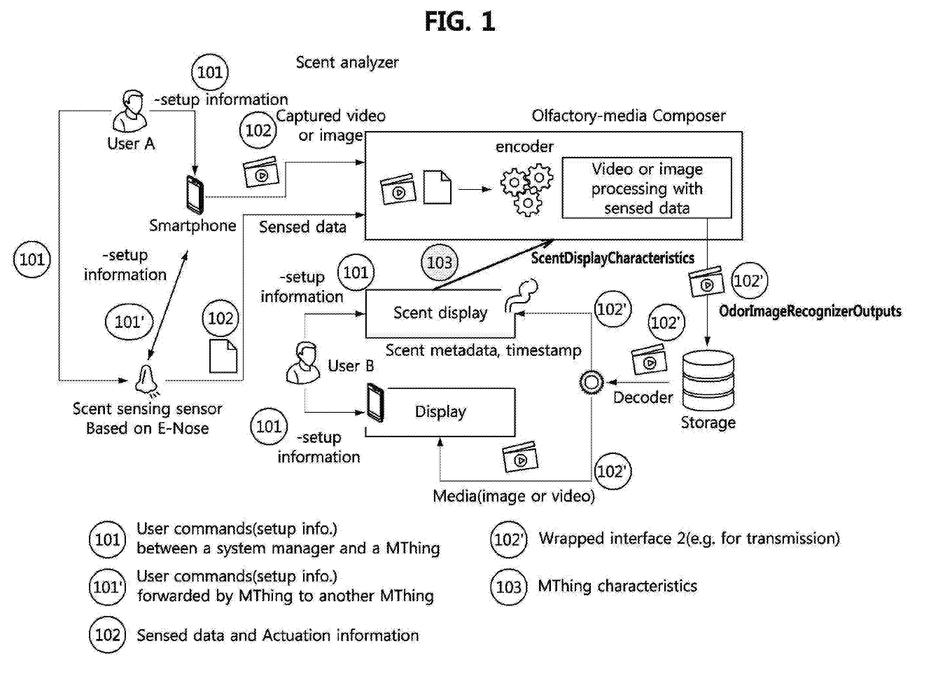

FIG. 1 is a view illustrating an overall execution environment including an olfactory information generator according to one embodiment of the present invention.

The olfactory information generator extracts an odor image from included multimedia content such as an image and describes the odor image in a data format sharable with a media thing. The olfactory information generator may extract an imagery component of a sense which is associated with a scent according to characteristics of the multimedia content. When the multimedia content includes a sound as a significant component, a sound which is associated with a particular scent may be extracted as an odor sound. For example, a meat-roasting sound may be classified as an odor sound which is associated with a scent of meat, and a fruit-cutting or cooking sound may be classified as an odor sound which is associated with a scent of fruit.

For convenience of description, a following description will focus on multimedia content with an emphasis on visual components such as a video and an odor image. However, the concept of the present invention is not limited to embodiments. The concept of the present invention described with respect to an odor image may be easily modified and applied to an odor sound or imagery component of another sense which is associated with a scent. For example, a component which generates label information and derives text-based information related to an odor image, odor sound, or imagery component of another sense which is associated with a scent may be applied to each and may also be applied to a post label information generation component or imagery components of a variety of senses.

The olfactory information generator according to one embodiment of the present invention may be a media thing which has a multimedia function. The olfactory information generator extracts an odor image capable of influencing olfactory senses by analyzing multimedia content and selects a scent component or a combination of scent components matching with characteristics of the odor image.

Referring to FIG. 1, User A or User B may input setup information into a smartphone, an E-nose gas sensor, a display apparatus, and an olfactory display (for example, a scent emitting device which interworks with a display) (101). The input of the setup information (101) is performed through an interaction between a system manager and media things, and the setup information is input into the media things by the system manager (101). Here, the smart phone, the E-nose gas sensor, the display apparatus, and the olfactory display may be referred to as the media things.

In FIG. 1, each of the media things may transmit and share the previously input setup information to and with the other media things (101′). For example, the previously input setup information may be transmitted and shared among the smartphone of User A, the E-nose gas sensor of User A, and the display and the olfactory display of User B (101′).

In FIG. 1, each of media things may generate sensed data or actuation information (102). For example, the E-nose gas sensor of User A may generate odor information (102) and the smartphone of the User A may generate video information (102).

One example of the olfactory information generator according to the present invention may be an olfactory-media composer shown in FIG. 1. The olfactory-media composer may extract a component related to an olfactory sense and a component capable of influencing the olfactory sense from multimedia content such as an image through searching and analyzing the multimedia content. Here, the extracted component may be an odor image. The odor image refers to an abstract image which is associated with a particular scent. Although image content has been generally described for convenience of description, the odor image is not limited to a visual component and may refer to all images related to the five human senses which are associated a particular scent. That is, a sound related to a particular scent and a tactile image related to a particular scent may be defined as odor images.

Here, the olfactory-media composer is shown as an independent apparatus in FIG. 1 but may be embodied as a part of other media things which do not deviate from the scope of the present invention such as a smartphone, an E-nose gas sensor, and the like.

Although one odor image may be representatively extracted from one piece of multimedia content, a plurality of components may complexly or individually/independently be associated with a scent. In this case, a plurality of odor images extracted from one piece of multimedia content may be represented as weighted representative data.

The extracted odor image may be transmitted to an apparatus capable of embodying olfactory information with the multimedia content, for example a scent emitting device. An olfactory display capable of being related to and synchronized with multimedia content to discharge a particular scent may embody the olfactory information. The extracted odor image may be, for example, transmitted to the olfactory display and synchronized with the multimedia content to be embodied such that multi-dimensional/multi-channel multimedia content including the olfactory information may be provided to a user.

The extracted odor image may be processed to be represented as text-based information. The odor image is evaluated and classified by a plurality of users or a trained group of experts and results thereof are described in order to be represented as text-based information related to the odor image. The text-based information may be referred to as tag information or label information related to the odor image.

The label information related to the odor image may include a source (related content) mark which refers to the multimedia content from which the odor image is obtained. The label information related to the odor image may competitively represent the concepts of a plurality of independent scents obtainable from one piece of content. Also, the label information related to the odor image may hierarchically represent abstract superordinate concepts and subordinate concepts related to one scent obtainable from one piece of content (for example, a smell of fruit->a smell of apples or a sweet smell->a smell of fruit).

A process of obtaining the tag information or label information related to the extract odor image may be performed through evaluation and classification by a plurality of users or a trained group of experts in early stages. When evaluation, classification, and technology information in early stages are collected, tag information or label information related to a similar or relevant odor image may be recognized based on pattern recognition. A process of recognizing label information of an odor image may be executed using an artificial intelligence (AI) machine learning technology.

An olfactory information generator according to another embodiment of the present invention synchronizes and stores odor information sensed by a gas sensor with multimedia content. Referring to FIG. 1, User A may obtain video information (102) through the smart phone. Meanwhile, User A may obtain the odor information (102) by using the E-nose gas sensor. The video information (102) obtained by the smart phone and the odor information (102) obtained by using the E-nose gas sensor may be synchronized and stored using the setup information (101) shared between the smart phone and the E-nose gas sensor. Instruction information related to the multimedia content (102) time-synchronized with the odor information 102 detected by the gas sensor may be stored with the odor information (102). A process of the olfactory-media composer as one example of the olfactory information generator may extract an odor image from the multimedia content (102) synchronized with the odor information (102) and may store the label information related to the odor image with respect to the odor information (102) detected by the gas sensor in a database or a memory. The odor information which occurs in the real world while synchronized with the multimedia content is managed so that the odor information interworks with the odor image included in the multimedia content such that multisensory multimedia content may be generated. In the real world, the odor information detected while synchronized with the multimedia content (for example, a video) is actual measurement data of the gas sensor related to the odor image in the multimedia content and may be utilized as reference data when the odor image and a scent component are matched or the label information is updated with respect to the odor image.

Here, the olfactory-media composer is shown as an independent apparatus in FIG. 1 but may be embodied as a part of other media things which do not deviate from the scope of the present invention such as a smartphone, an E-nose gas sensor, and the like.

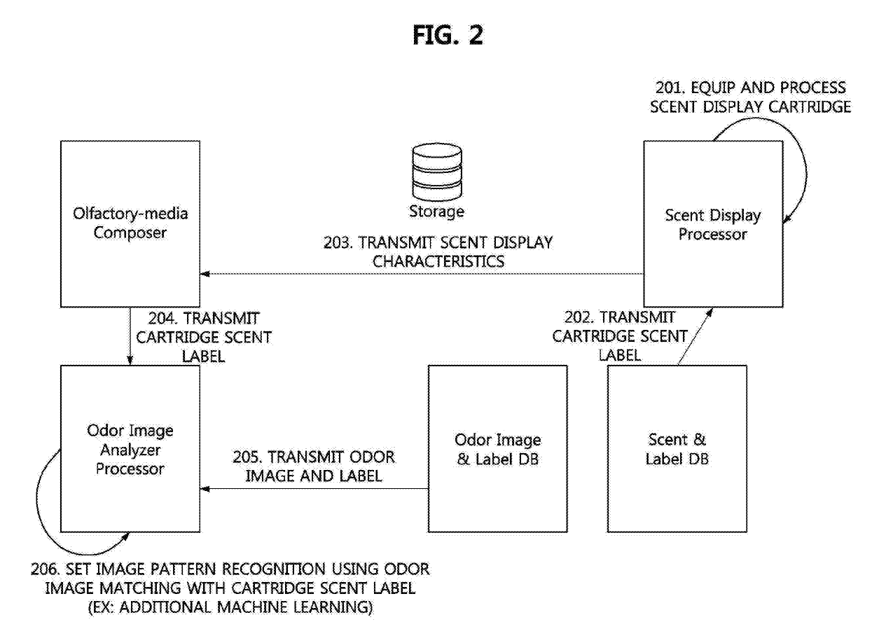

Referring back to FIG. 1, characteristics of the scent component possessed by the olfactory display may be defined as characteristics (103) related to olfactory sensation of a media thing. The olfactory information generator analyzes and processes the characteristics (103) related to the olfactory sensation of the media thing. The processed information is transmitted back to and shared by the media thing via a wrapped interface (102′) for data transmission or sharing. For media things, particularly the scent emitting device, it is general to use a plurality of scent components equipped in a scent cartridge. The scent component has individual characteristics and corresponds to a particular domain. Representation of the particular domain to which the scent component corresponds in a language intuitively recognizable by a human being is the label information of the scent component. FIG. 2 illustrates a process of obtaining characteristic information possessed by the scent component of the scent cartridge of the scent emitting device as the label information.

FIG. 2 is a view illustrating a process of analyzing or recognizing an odor image matched with a scent component equipped in a scent cartridge and generating and updating label information which represents the scent component.

The olfactory-media composer, as one example of the olfactory information generator of the present invention, obtains text-based label information related to the scent component included in the scent cartridge. Here, when the text-based label information related to the scent component does not exist, the text-based label information may be generated by analyzing odor information of the scent component. When the odor information of the scent component is analyzed, odor information when the scent component is actually discharged may be collected by using the gas sensor such as the E-nose and the like. With respect to the collected odor information, an odor image may be extracted and label information related to the odor image may be obtained by searching a previously analyzed odor information-odor image association database to generate label information related to the scent component.

In another embodiment, it may be assumed that text-based label information related to a scent component is input by a user. Here, the label information related to the scent component may not be identical to generally used label information related to the odor image. The olfactory information generator may collect label information highly related to the label information input by the user and the label information related to the odor image related to the scent component through analyzing syntax of a text. The olfactory information generator may store the label information input by the user related to the scent component and label information (generalized, standardized, or previously collected label information) derived through executing pattern recognition, database searching, and syntax analysis of the text together in the memory or the database.

When the label information input by the user related to the scent component does not coincide with the label information of the odor image of the multimedia content which is to be provided to the user, the olfactory information generator may match the label information input by the user related to the scent component with the label information of the odor image of the multimedia content by using the label information derived through pattern recognition, searching the database for the label information of the odor image related to the scent component, and analyzing the syntax of the text.

With respect to first label information of the scent component, which is derived first by analyzing the scent component, the olfactory information generator may obtain second label information of the scent component which is updated periodically whenever a particular event (a user command, addition of multimedia content data, and addition of an odor image database) occurs through pattern recognition, searching the database, and analyzing the syntax of the text.

A processor of the scent display shown in FIG. 2 is an olfactory information generator according to still another embodiment of the present invention. The scent components mounted on the scent cartridge of the scent display are recognized (201). Scent display characteristic information (203) initially recognized related to the scent component is transmitted to the olfactory-media composer. Here, the characteristic information (203) is one example of the characteristic information (103) of the scent emitting device shown in FIG. 1. The characteristic information (203) may be first label information input by the user or may be odor information (quantitative gas detection information) of the gas sensor obtained through supplementation by the E-nose gas sensor when there is no label information input by the user, depending on the embodiment. The olfactory-media composer may generate cartridge scent label information (204) and the cartridge scent label information (204) to an odor image analyzer processor. The odor image analyzer processor may update the cartridge scent label information (204) through image pattern recognition using an odor image matching with the cartridge scent label information (204) and may add further standardized or generalized label information. The image pattern recognition using the odor image may be set to be performed through additional machine learning.

As one embodiment of the olfactory information generator of the present invention, the processor of the scent display may transmit a search query related to a particular scent component to a scent & label database, and prestored cartridge scent label information (202) may be transmitted from the scent & label database to the processor of the scent display. Meanwhile, an odor image & label database may transmit an odor image and label information corresponding to the odor image in response to a search query of the odor image analyzer processor.

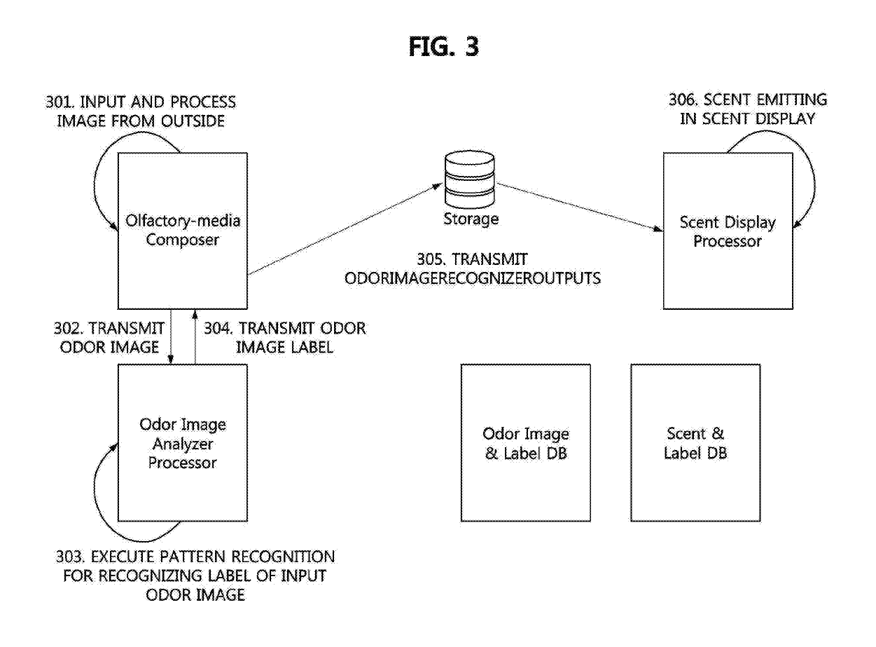

FIG. 3 is a view illustrating, as an example of a process of embodying the scent display or the olfactory display, a process of extracting an odor image which is associated with a scent from image content, representing the odor image as label information, and sharing the label information with the scent display or the olfactory display and emitting a scent.

Referring to FIG. 3, the olfactory-media composer receives and processes external image content that has been input (301). The olfactory-media composer extracts an odor image from the image content. The extracted odor image is transmitted to the odor image analyzer processor (302). The odor image analyzer processor may perform pattern recognition for recognizing a label of an input odor image. The label information of the odor image, recognized by the odor image analyzer processor, is transmitted back to the olfactory-media composer (304).

The olfactory-media composer transmits OdorImageRecognizerOutputs, which is standardized label information, to a storage through the wrapped interface for data transmission and sharing (305). OdorImageRecognizerOutputs, which is the standardized label information stored in the storage, is transmitted to the processor of the olfactory display (305), and the olfactory display performs a scent-emitting treatment which interworks with the image content by controlling scent emission in the olfactory display in order to discharge a scent component or a combination of a plurality of scent components equipped in the scent cartridge of the olfactory display by using the label information of the odor image of the transmitted multimedia content (306).

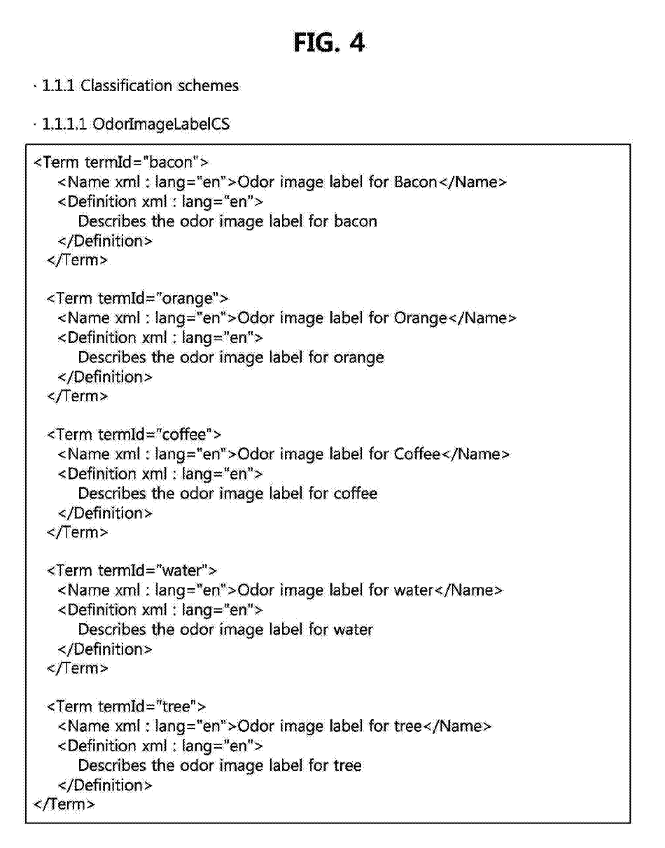

FIG. 4 is a view illustrating an example of a data format which represents label information of an odor image.

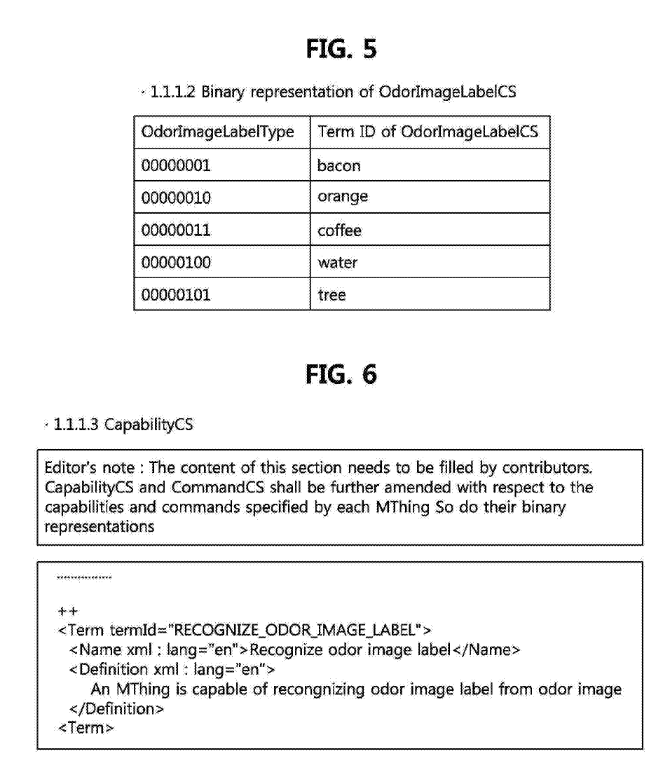

FIG. 5 is a view illustrating an example of a binary representation of the label information of FIG. 4.

Referring to FIG. 4, a data format and a syntax structure which represent label information of an odor image in an XML format-language are illustrated. As shown in FIG. 4, an odor image may be embodied as a word which is associated with a particular category or a characteristic scent and has representativeness. Bacon, orange, coffee, water, tree, and the like are associated with particular scents and may suggest unique atmospheres thereof. For example, bacon may allude to an atmosphere of “during a meal,” orange may allude to something being sweet and fragrant, coffee may allude to an atmosphere of rest or talk, water may allude to something being fresh and healthy, and tree may allude to something being fresh and to an image of nature.

As described above, label information related to a particular odor image may be represented, and additional label information related to an abstract superordinate concept suggested by the label information may be added.

Otherwise, a plurality of superordinate concepts related to one odor image may be competitively listed. For example, since orange may be connected to a superordinate concept such as “fruit” and an abstract concept such as “sweet,” the orange may be connected to the above keywords.

Semantic similarity or semantic relation among the keywords of the odor image may be obtained by applying a natural language processing principle and may be further specified and diversified by artificial intelligence-based machine learning.

Referring to FIG. 5, it is shown that each piece of the label information of the odor image such as bacon, orange, coffee, water, tree, and the like may be encoded by a series of binary numbers.

FIG. 6 is a view illustrating an example of representative data which represents whether the olfactory information generator or the media thing (Mthing) has a function of recognizing label information of an odor image from the odor image.

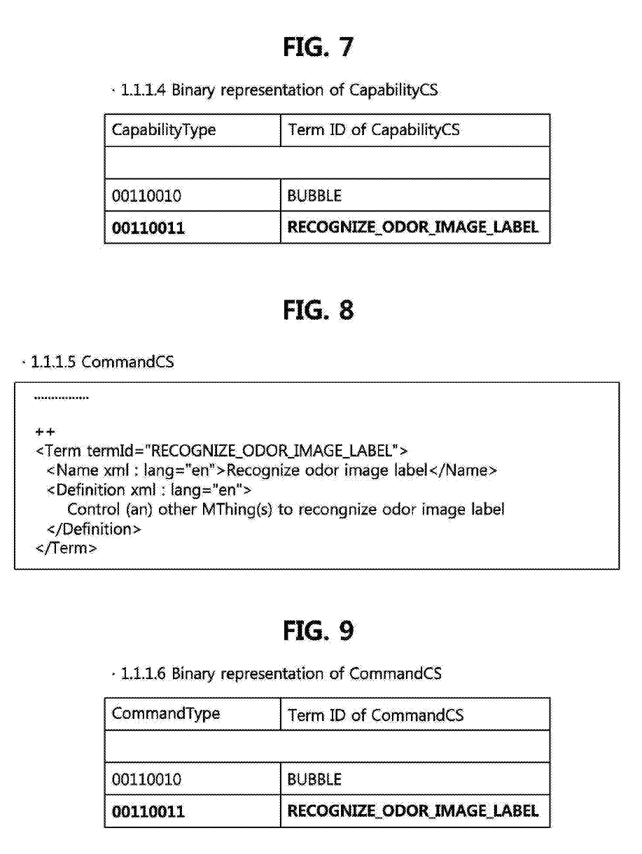

FIG. 7 is a view illustrating an example of a binary representation of the representative data of FIG. 6.

Referring to FIG. 6, there is illustrated one example of representative data and a data-syntax structure related to whether a media thing only simply manages sensor-level information related to an odor image or also possesses a function of recognizing label information of the odor image. In FIG. 7, it is shown that the representative data may be encoded by using a series of binary numbers.

FIG. 8 is a view illustrating an example of representative data which represents a command for controlling (an)other media thing(s) so that the other media thing(s) allow(s) the olfactory information generator or the media thing(s) to recognize label information of an odor image.

FIG. 9 is a view illustrating an example of a binary representation of the representative data of FIG. 8.

Referring to FIG. 8, there is illustrated one example of representative data and a syntax structure of a command for controlling (an)other media thing(s) so that the other media thing(s) allow(s) the media thing(s) to recognize label information of an odor image. It may be assumed that the media thing(s) controlled by the command of FIG. 8 has a function of recognizing the label information shown in FIG. 6.

Referring to FIGS. 6, 8, and 3, the control command of FIG. 8 may be transmitted from the olfactory-media composer to the odor image analyzer processor. Here, the control command may be transmitted with the odor image (302). The function of recognizing label information of an odor image in FIG. 6 may be used to describe a function of the odor image analyzer processor of FIG. 3.

Although one example in which the olfactory-media composer and the odor image analyzer processor are distinguished from each other is shown in FIG. 3, depending on an embodiment, the olfactory-media composer and the odor image analyzer processor may be embodied as one processor.

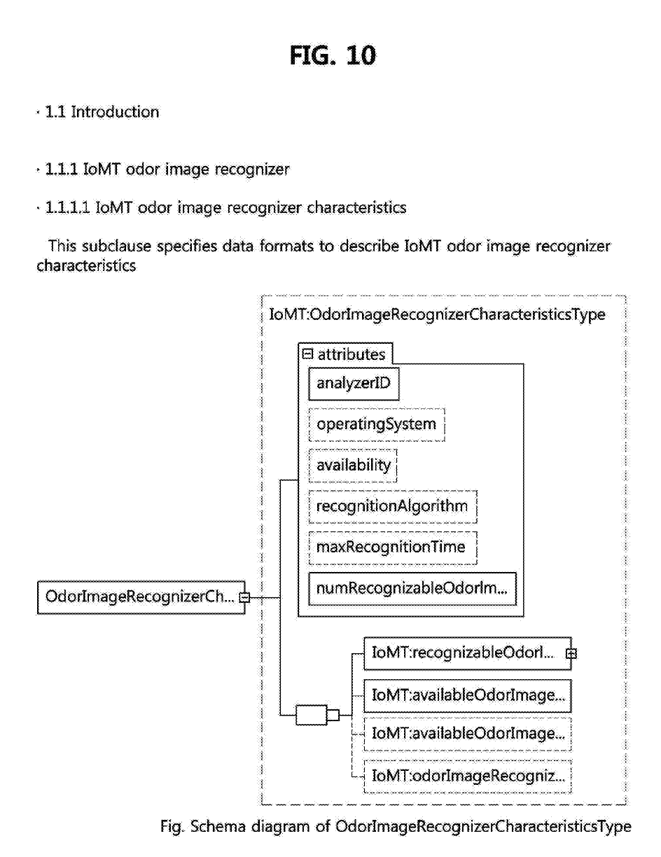

FIG. 10 is a view illustrating an example of a schema diagram of representative data which represents a recognition function of a processor, the olfactory information generator, or a media thing which recognizes an odor image or label information of the odor image.

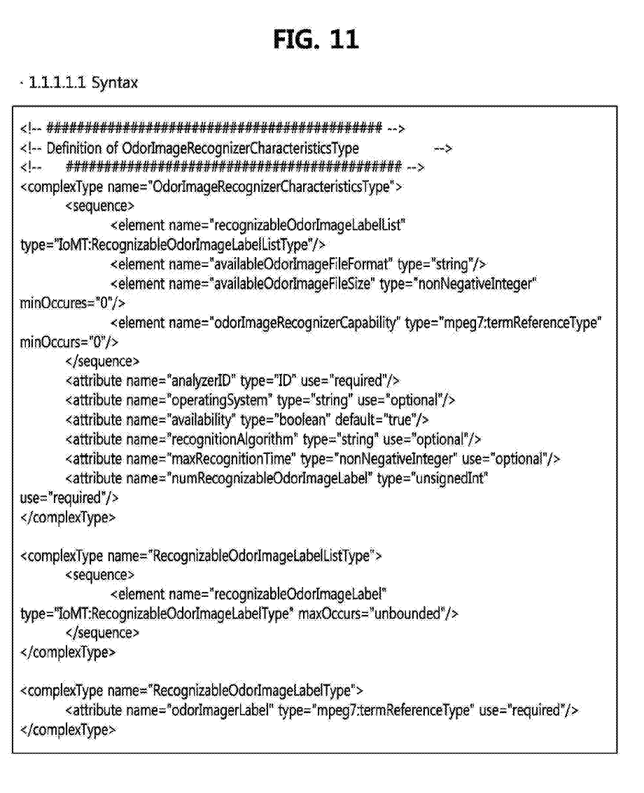

FIG. 11 is a view illustrating an example of a syntax structure of representative data which represents a recognition function of a processor, the olfactory information generator, or a media thing, which recognizes an odor image or label information of the odor image.

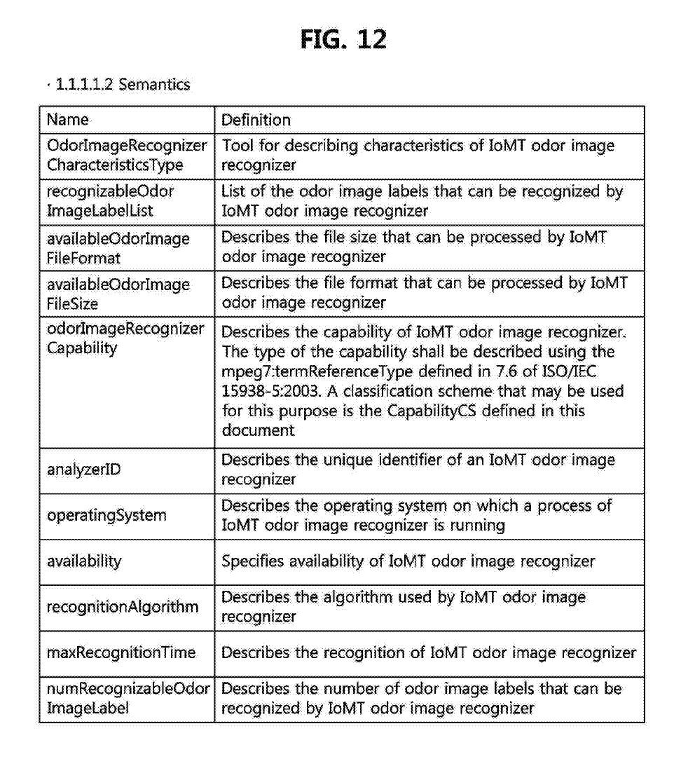

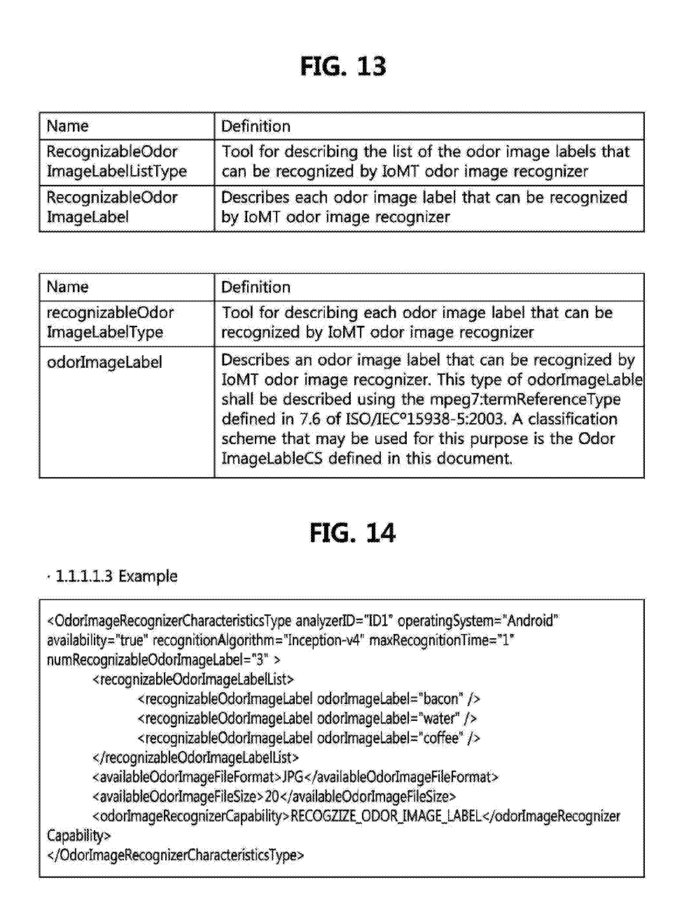

FIGS. 12 and 13 are views illustrating semantics of representative data which represents a recognition function of recognizing an odor image or label information of the odor image.

FIG. 14 is a view illustrating an example of representative data to which the syntax structure of FIG. 11 and the semantics of FIGS. 12 and 13 are applied.

Referring to the schema diagram of FIG. 10 and the syntax structure of FIG. 11, representative data which represents a recognition function of a media thing may include a recognizable odor image label list, an available odor image file format, an available odor file size, odor image recognizer capability, and the like. A description of the data in a subfield is illustrated through the illustration of the semantics of FIGS. 12 and 13.

Referring to FIG. 14, there is illustrated a function of a media thing capable of recognizing a label of an odor image related to three concepts of bacon, water, and coffee by applying the syntax structure of FIG. 11.

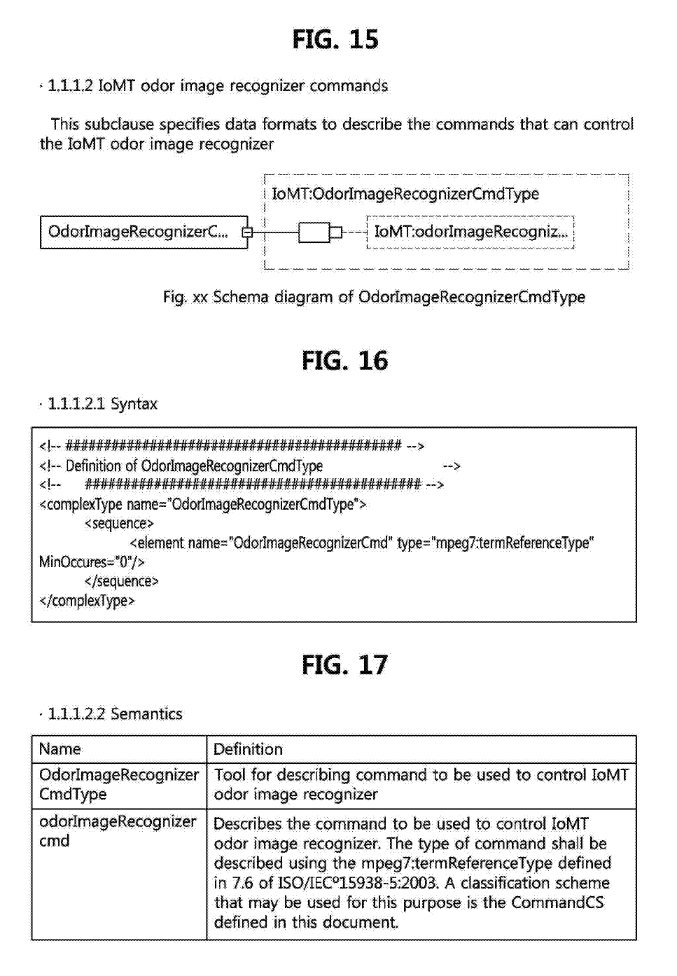

FIG. 15 is a view illustrating an example of a schema diagram of representative data which represents a recognition command for a processor, the olfactory information generator, or a media thing which recognizes an odor image or label information of the odor image.

FIG. 16 is a view illustrating an example of a syntax structure of representative data which represents a recognition command for a processor, the olfactory information generator, or a media thing which recognizes an odor image or label information of the odor image.

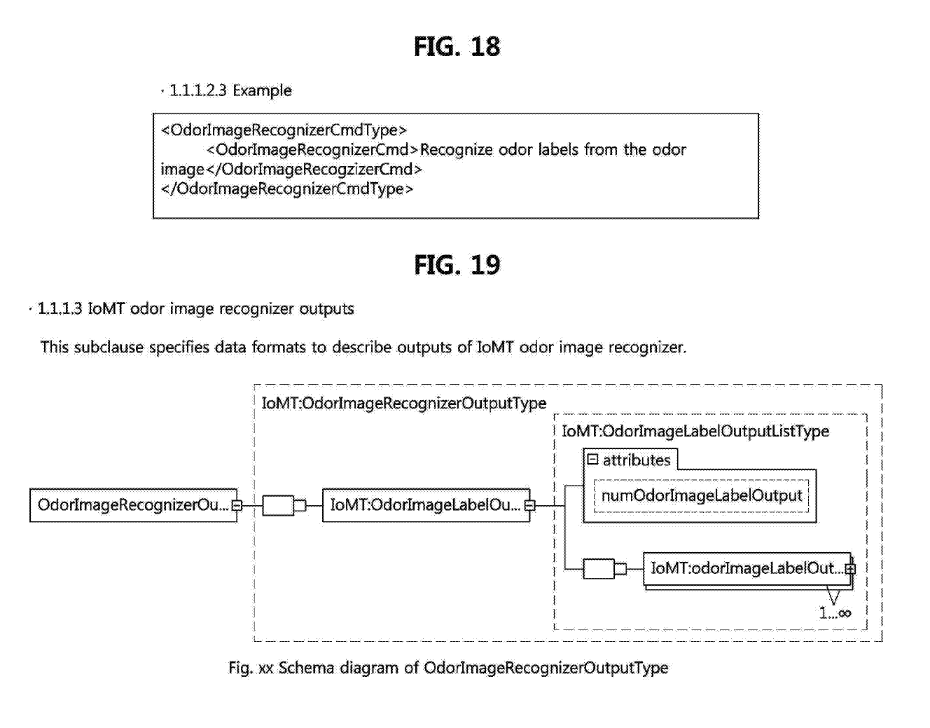

FIG. 17 is a view illustrating example semantics of representative data which represents a recognition command for a processor, the olfactory information generator, or a media thing which recognizes an odor image or label information of the odor image.

FIG. 18 is a view illustrating an example of representative data to which the syntax structure of FIG. 16 and the semantics of FIG. 17 are applied.

Referring to the schema diagram of FIG. 15, an odor image recognition command may be embodied as a data field of a lower hierarchy of an odor image recognition function. The syntax structure of FIG. 16 has a close relation to representative data of a label recognition command shown in FIG. 8.

FIG. 19 is a view illustrating an example of a schema diagram of representative data which represents a result of recognizing an odor image or label information of the odor image using the olfactory information generator or a media thing.

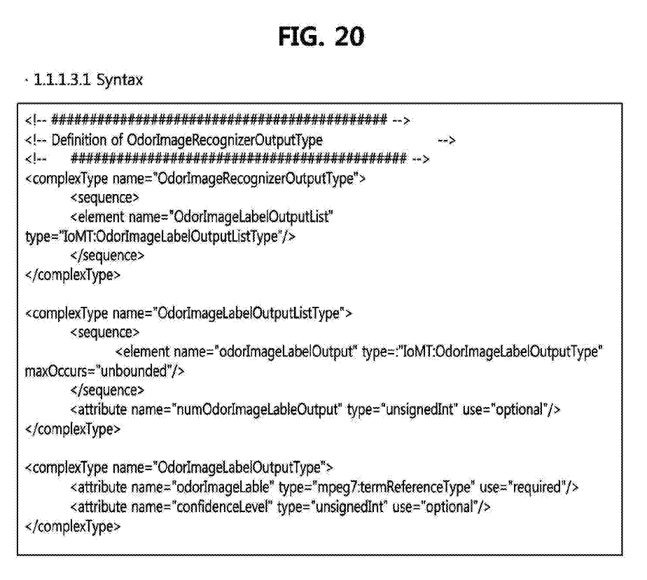

FIG. 20 is a view illustrating an example of a syntax structure of representative data which represents a result of recognizing an odor image or label information of the odor image using the olfactory information generator or a media thing.

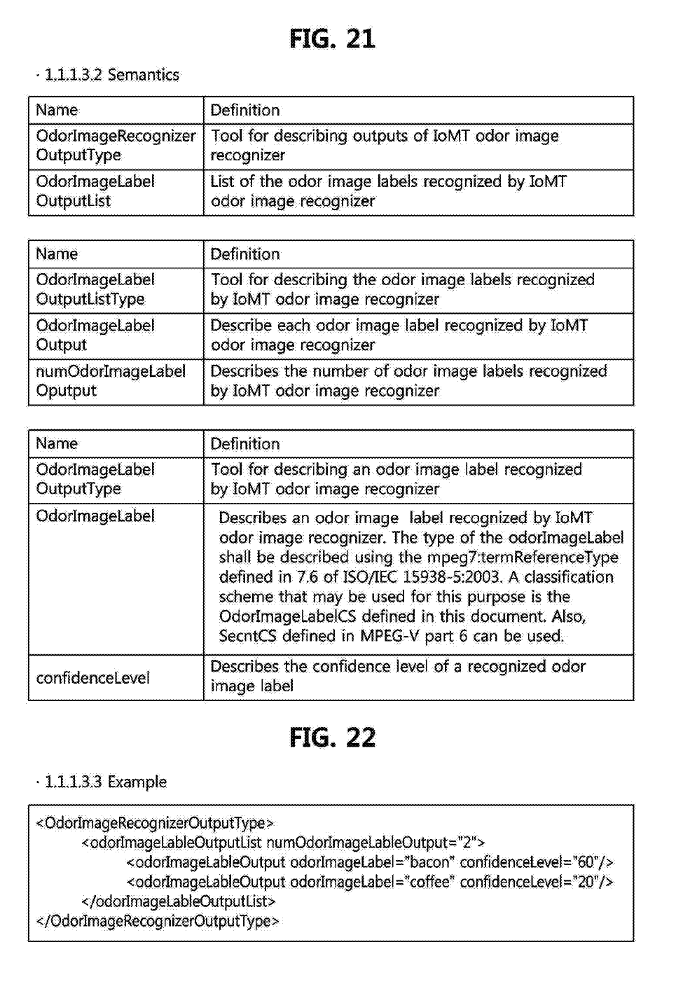

FIG. 21 is a view illustrating example semantics of representative data which represents a result of recognizing an odor image or label information of the odor image by the olfactory information generator or a media thing.

FIG. 22 is a view illustrating an example of representative data to which the syntax structure of FIG. 20 and the semantics of FIG. 21 are applied.

Referring to FIGS. 20 to 22, label information of an odor image, obtained from an analysis result, may additionally include confidence level information on the analysis.

FIG. 22 illustrates a case in which bacon is detected as odor image label information with a confidence level of 60 and coffee is detected as odor image label information with a confidence level of 20. Although the confidence level is shown as a main parameter for convenience of description, influence, contribution level, and importance of a particular component among a plurality of odor images included in one image content may be evaluated and added as parameters. Otherwise, a relative strength of impression on an olfactory sense may be evaluated for each of the plurality of odor images of the image content and may be added as a parameter. Otherwise, a relation/suitability level with a particular scent component capable of emitting a scent in relation to the olfactory display may be evaluated with respect to the plurality of odor images shown in the image content and may be represented in a form of an evaluation indicator of the IoMT field.

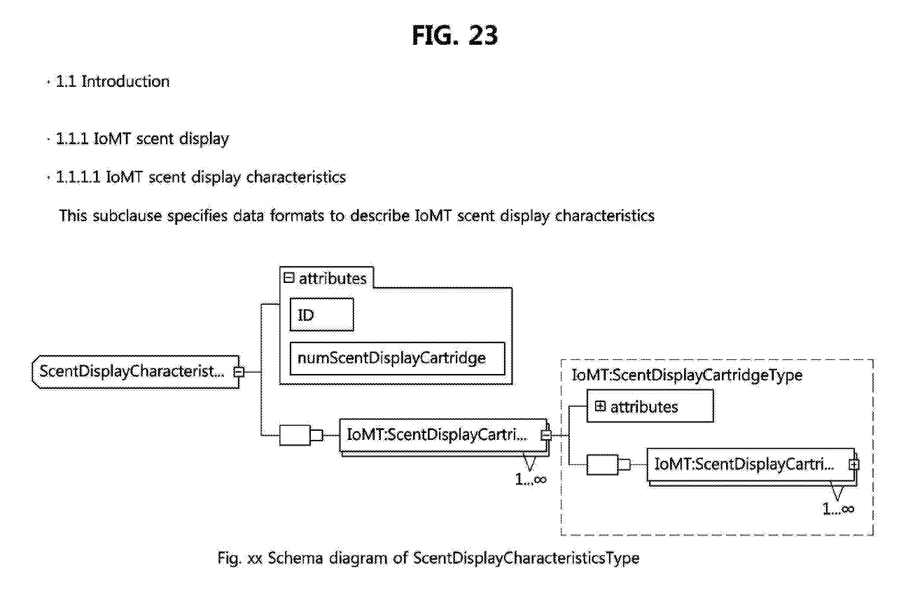

FIG. 23 is a view illustrating an example of a schema diagram of representative data handled in a system environment including the olfactory display or the scent display.

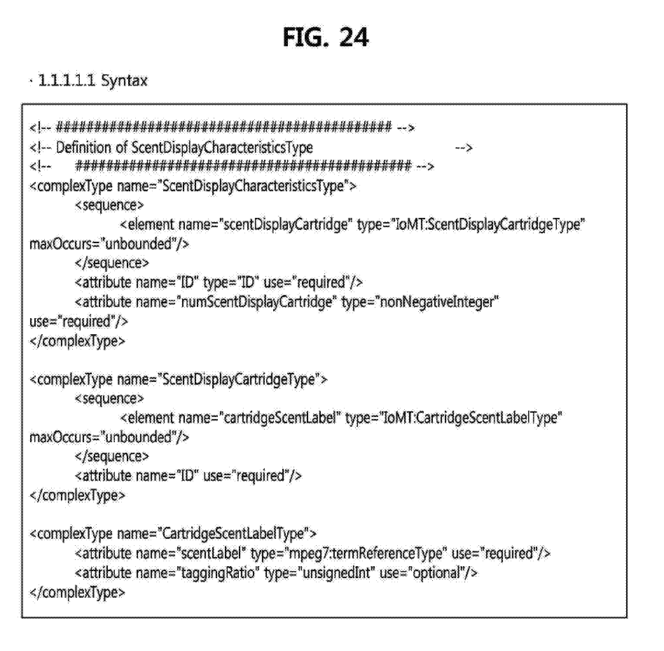

FIG. 24 is a view illustrating an example of a syntax structure of representative data handled in a system environment including the olfactory display or the scent display.

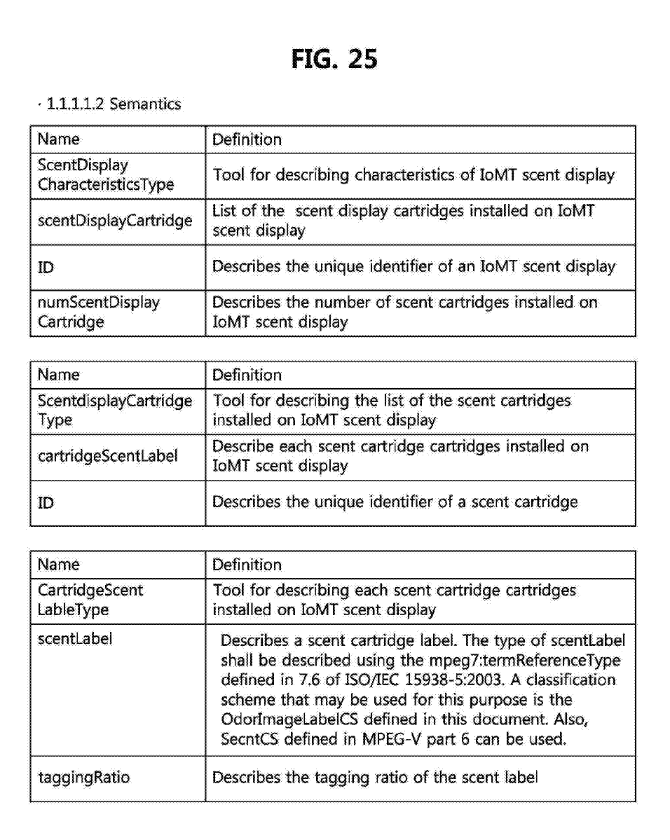

FIG. 25 is a view illustrating an example of semantics of representative data handled in a system environment including the olfactory display or the scent display.

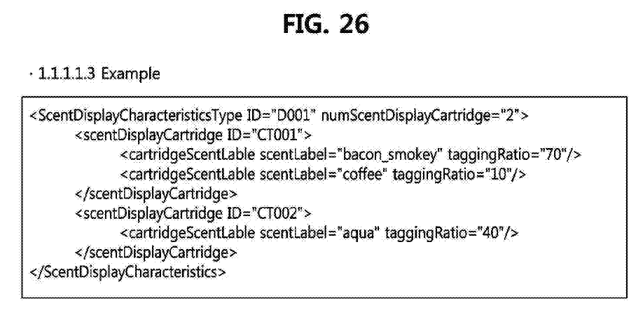

FIG. 26 is a view illustrating an example of representative data to which the syntax structure of FIG. 24 and the semantics of FIG. 25 are applied.

Referring to FIGS. 23 to 26, information on characteristics of the scent cartridge and representative data which represents label information of the scent components included in the scent cartridge are introduced as a significant field of data of the representative data handled in the system environment including the olfactory display or the scent display.

Also, since even the same scent component may have different imagery components related to a scent recognized by a human being due to a tagging ratio thereof, the representative data handled in the system environment including the olfactory display or the scent display may include scentLabel and tagging ratio as data fields.

Here, the tagging ratio may be applied as a concept corresponding to a concentration of gas or may be applied as a concept corresponding to a strength defined through evaluation by a plurality of users or a trained expert. That is, although an example in which the tagging ratio has a certain value is shown in FIG. 26, the tagging ratio is not definitively represented as a value but may be represented as a relative grade after a quantitative evaluation.

In FIGS. 1 to 3, there is illustrated a user scenario in which the olfactory-media composer operates as an independent apparatus separated from other media things such as the smartphone, the olfactory display, and the like. However, the concept of the present invention is not limited thereto and may be embodied by embodying and executing the olfactory-media composer in a form of an application program executed in the smartphone. In this case, the olfactory-media composer, that is, the processor in the olfactory information generator, may be an application processor of the smart phone.

When the olfactory information generator (the olfactory-media composer) is embodied as a separate media thing, the olfactory information generator may include a processor, a memory, a storage, and a communication module. The processor may perform functions of extracting an odor image, recognizing label information of the odor image (or transmitting a command to another media thing for recognition), and the like. Necessary information may be stored in a memory or a storage, and a communication module may be included for communication and sharing with other media things.

In still another embodiment, a processor included in the olfactory display (including the scent emitting device) may operate as the olfactory information generator. The olfactory information generator may further include a memory, a storage, and a communication module in addition to the processor.

The embodiments of the present disclosure may be implemented as program instructions executable by a variety of computers and recorded on a computer readable medium. The computer readable medium may include a program instruction, a data file, a data structure, or a combination thereof. The program instructions recorded on the computer readable medium may be designed and configured specifically for the present disclosure or can be publicly known and available to those who are skilled in the field of computer software. Examples of the computer readable medium may include magnetic media such as hardware disk, floppy disk, and magnetic tape, optical media such as CD-ROM and DVD, magneto-optical media such as floptical disk, ROM, RAM, and flash memory, which are specifically configured to store and execute the program instructions. Examples of the program instructions include machine codes made by, for example, a compiler, as well as high-level language codes executable by a computer, using an interpreter. The above exemplary hardware device can be configured to operate as at least one software module in order to perform the embodiments of the present disclosure, and vice versa.

While the example embodiments of the present invention and their advantages have been described in detail, it should be understood that various changes, substitutions and alterations may be made herein without departing from the scope of the invention.

What is claimed is:

1. An olfactory information generator which generates olfactory information sharable between the real world and at least one virtual world, the olfactory information generator comprising a processor,
wherein the processor receives multimedia content, extracts an odor image or an odor sound included in the multimedia content, and generates representative data related to the odor image or the odor sound by describing information on the extracted odor image or odor sound in a data format sharable by a media thing.

2. The olfactory information generator of claim 1, wherein the processor analyzes the extracted odor image or odor sound and generates text-cased label information capable of describing an odor of the odor image or the odor sound through a semantic evaluation or an abstract process related to the analyzed odor image or odor sound.

3. The olfactory information generator of claim 2, wherein the processor updates the label information of the extracted odor image or odor sound by applying a pattern recognition technique to odor image or odor sound data included in a database related to the extracted odor image or odor sound.

4. The olfactory information generator of claim 1, wherein the processor extracts each of a plurality of odor images or odor sounds included in the multimedia content and generates the representative data by using information on each of the plurality of extracted odor images or odor sounds, with a weight.

5. The olfactory information generator of claim 1, wherein the processor generates the representative data by using synchronization information between the extracted odor image or odor sound and the multimedia content to form a scent emitting sequence corresponding to the odor image or the odor sound to be synchronized with execution of the multimedia content.

6. The olfactory information generator of claim 1, wherein the processor receives sensory information related to a scent in the real world, which is generated by a gas sensor, extracts odor image or odor sound information related to content of the multimedia content, which is time-synchronized with the sense information, and generates the representative data by adding the sensory information to the extracted odor image or odor sound information extracted related to the content time-synchronized with the sensory information.

7. An olfactory information generator which generates olfactory information sharable between a real world and at least one virtual world, the olfactory information generator comprising a processor,
wherein the processor obtains text-based label information related to a scent component included in the scent cartridge and generates representative data related to the label information by describing information on the label information related to the scent component in a data format sharable by a media thing.

8. The olfactory information generator of claim 7, wherein the processor searches an odor information-odor image association database, an odor information-odor sound association database, or an odor information-label information association database for the scent component and extracts the label information corresponding to the scent component.

9. The olfactory information generator of claim 7, wherein the processor obtains the label information by a user input, extracts modified label information corresponding to the label information by searching an odor information-odor image association database, an odor information-odor sound association database, or an odor information-label information association database for the label information, and generates the representative data in connection with the label information and the modified label information.

10. The olfactory information generator of claim 7, wherein the processor, periodically or when a particular event occurs, executes searching of an odor information-odor image association database, an odor information-odor sound association database, or an odor information-label information association database, executes pattern recognition or text syntax analysis, and updates the label information.

11. A method of generating olfactory information, in which olfactory information sharable between a real world and at least one virtual world is generated, the method comprising:
receiving multimedia content;
extracting an odor image or an odor sound included in the multimedia content; and
describing information on the extracted odor image or odor sound in a data format sharable by a media thing.
